(19) United States
(12) Patent Application Publication
(10) Pub. No.: US 2009/0060995 A1
(43) Pub. Date: Mar. 5, 2009

(54) Dispersible sustained release pharmaceutical compositions

(76) Inventors: Kamalinder Kaur Singh, Mumbai (IN); Madhiri Dahiwal, Mumbai (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 11/330,522

(22) Filed: Jan. 12, 2006

(30) Foreign Application Priority Data
Jan. 13, 2005 (IN) 880/MUM/2004

(51) Int. Cl. A61K 9/22

(52) U.S. Cl. 424/465

(57) ABSTRACT

Disclosed herein are pharmaceutical compositions, in particular, dispersible compositions comprising sustained release granules of at least one active pharmaceutical ingredient and at least one release retard hydrophobic polymer formulated with super disintegrant and lubricant, to achieve dispersible and sustained release effect.

### UNITED STATES GOVERNMENT INTEREST

None.

### RELATED APPLICATIONS

This application claims priority from India patent application Serial No. 880/MUM/2004, which has an effective filing date of 13 Jan. 2005.

### FIELD OF THE INVENTION

The present invention relates to pharmaceutical compositions, in particular, dispersible compositions comprising sustained release granules of at least one active pharmaceutical ingredient and at least one release retard hydrophobic polymer formulated with super disintegrant and lubricant, to achieve dispersible and sustained release effect.

### BACKGROUND OF THE INVENTION

Certain medical conditions for example chronic pain, infections etc, require the administration of a pharmaceutical in such a way that its release is sustained over an extended period of time. This is achieved by repeated administration of an immediate release formulation of the required drug at frequent predetermined intervals. In such cases, sustained release formulations are preferred to be administered, which release the drug at predetermined time intervals, thus eliminating the need of repeated administration.

Effervescent and water dispersible dosage forms are preferred as they are convenient to administer especially to the pediatric patients or those who face difficulty in swallowing solid dosage forms.

The Pharmaceutical Journal, Mar. 12, 1983, p 289-294, F. E. J. Sendall et al. discloses Effervescent and water-dispersible tablets which are know well known. However, prior to this it was not suggested that granular sustained-release formulations could be presented successfully in the form of effervescent or water-dispersible tablets.

However, subsequently research work was carried out to get formulations in the form of dispersible tablet with a sustained release drug profile. The prior art for the same is mentioned below.

EP0313328 describes a granular sustained-release formulation of a pharmacologically active substance presented in the form of a tablet. The said tablet comprises a core containing active ingredient with one or more excipients which is further coated with water insoluble but water swellable acrylic polymer and a water soluble hydroxylated cellulose derivative, thus providing sustained-release profile on administration to patients. In the said formulation the mannitol is used as water dispersing agent as a result of which the tablet disperses in the mouth.

EP0052075 describes a granular delayed-release form of pharmaceutical active substances, which contains a granulated or crystalline pharmaceutical active substance coated with coating materials retarding the release of the active substances, these coating materials consist of a homogeneous mixture of a polyacrylate insoluble but dispersible in water and a cellulose ether insoluble but dispersible in water.

U.S. Pat. No. 4,988,679 discloses an orally ingestible liquid composition for suspending therein an orally administrable pharmaceutically active composition releasable over an extended period of time which comprises triglyceride of a medium chain length alkanoic acid or distilled acetylated monoglycerides, a liquid, high HLB polyglyceryl ester, and colloidal silicon dioxide together with a material soluble or dispersible therein and capable of being insolubilized by a pharmaceutically acceptable polyvalent cation and a solid pharmaceutically acceptable salt containing the cation required therefore. Sustained release compositions based thereon containing pharmaceutically active agents are also disclosed

U.S. Pat. No. 5,780,055 describes cushioning beads comprising microcrystalline cellulose and a disintegrant (preferably croscarmellose sodium). The cushioning beads are prepared by extrusion-spheronization, followed by freeze-drying technique. This patent has been disclosed water-dispersible tablets having high tensile strength, comprising the cushioning beads and biologically active ingredient-loaded beads, wherein optionally, the tablets can contain a viscosity enhancer in the form of separate beads, or as a component of the biologically active ingredient-loaded beads. All these beads are prepared by extrusion-spheronization technique followed by freeze drying technique. Further, all these beads are blended and compressed to obtain a water dispersible tablet. The above prior art is very difficult and tedious to practice, thus, leaving ample scope to carry out further investigations.

Hence, the present invention is aimed to provide a cost effective, simple and industrially viable process to prepare dispersible sustained release compositions which are suitable particularly to pediatric and geriatric patients. Further the present formulation may be dispersed in water prior to administration. On the other hand the present formulation comprises granules made by wet granulation which are not coated like those mentioned in prior art.

### OBJECTIVES OF THE PRESENT INVENTION

The objective of the present invention is to ameliorate one or more of the problems associated with prior art.

Another objective of the present invention is to provide a dispersible sustained release tablet composition which disintegrate rapidly in water, and form a homogenous suspension which can be easily swallowed by children and the elderly, with minimal effect on the release properties of the biologically active ingredient.

Further object of the present invention is to provide sustained release drug granules which are able to sustain the release of the active ingredient over a period of 12 hrs in the gastro intestinal tract.

Still another objective of the invention is to reduce the frequency of administrations and to provide for convenience of administration to geriatric and pediatric patients.

### SUMMARY OF THE INVENTION

The present invention discloses pharmaceutical compositions, in particular, dispersible compositions comprising sustained release granules of at least one active pharmaceutical ingredient and at least one release retard hydrophobic polymer formulated with super disintegrant and lubricant, to achieve dispersible and sustained release effect. Further, being in the solid dosage form the antibiotics are more stable in the said formulation.

### DETAILED DESCRIPTION OF THE INVENTION

The dispersible sustained release tablet composition of the present invention is suitable for oral administration to pediatric and geriatric patients as it is easy to swallow and also provides sustained release of the active pharmaceutical ingredient from the granules, thus reducing the frequency of administration.

According to the present invention, the dispersible sustained release tablet compositions comprises sustained release granules of at least one active pharmaceutical ingredient and at least one release retard hydrophobic polymer, a super disintegrant, lubricant and other optional conventional agents such as coloring agents, flavoring agents and sweetening agents.

The composition of the present invention comprises at least one antibiotic along with other inert pharmaceutical excipients. The antibiotics are selected from the group of β-lactams, cephalosporins and/or penicillins.

In the preferred embodiment, the antibiotic is amoxicillin, in the form of amoxicillin-trihydrate. In the present invention, the pediatric dose of 200 mg of amoxicillin to an adult dose of up to 1000 mg tablets were prepared to deliver in a sustained release tablet composition at an interval of about 12 hours.

In another preferred embodiment, the antibiotic is cefixime in the form of cefixime trihydrate suitably formulated to achieve the dispersible and sustained effect.

The release retarding polymers used in the said formulation may be a hydrophobic polymer is selected from group consisting of methyl cellulose, ethylcellulose, Carbomers Eudragits, Hydroxy propyl methyl cellulose, Hydroxypropyl cellulose or Hydroxyethyl cellulose. One preferred hydrophobic polymer is ethylcellulose.

The ratio of the active pharmaceutical ingredient and ethylcellulose in the sustained release granules are in the range of 1:3. The active:ethylcellulose ratio may be vary from 1:0.5 or 1:0.75 or 1:1 or 1:2 or 1:3, most preferably 1:0.75

The super disintegrants used may be croscarmellose sodium, crospovidone, sodium starch glycolate, and starch. The amount of super disintegrant may be 15 to 25% of tablet weight. One preferred super disintegrant is sodium starch glycolate.

The lubricant is selected from the group of metallic silicates, metallic stearates or colloidal silicon dioxide (Aerosil), starch, talc, micronized amorphous silica or amorphous silica. A lubricant employed may preferably be Aerosil. The amount of lubricant may be 10 to 15% of tablet weight.

The tablet formulation may also include other conventional excipients such as coloring agents, flavouring agents and sweeteners.

Suitable coloring agents for use in the formulation preferably include erythrosine at an amount of 0.3 to 0.8% of tablet weight.

Suitable flavouring agents for use in the formulation include strawberry, tutti-fruti, preferably strawberry at an amount of 0.3 to 0.8% of tablet weight.

Suitable sweetening agents for use in the formulation include saccharin sodium at an amount of 1.0 to 3.0% of tablet weight.

The sustained release granules of active ingredient were prepared by the wet granulation technique.

### Procedure for Preparation of Sustained Release Granules by Wet Granulation Technique:

The active ingredient was mixed with ethylcellulose and coloring agent in geometric proportion. Mixing was continued further for 1 hour so as to ensure uniform mixing. To the uniform mixture obtained, distilled ethanol was added slowly, so as to form dough which was then passed through sieve no. 16 and the granules obtained were air-dried.

### Procedure for Preparation of the Sustained Release Dispersible Tablets:

The glidant, Aerosil was sieved through sieve no. 85 to break any lumps. The color, erythrosine, was also sieved through sieve no. 85. The glidant and the color were then mixed intimately and again sieved through sieve no. 85 so as to get a uniform distribution of the color in the glidant.

The flavor strawberry/tutti-fruti, was separately sieved through sieve no. 85 and was mixed with the color and glidant mixture.

The sweetener sodium saccharin was crushed in a mortar and passed through sieve no. 45 and was mixed evenly with the above mixture of color, flavor and glidant mixture.

To the above mixture of color, flavor, glidant and sweetener, the disintegrant was added and mixed thoroughly. The disintegrant used maybe crospovidone, croscarmellose sodium or starch, preferably sodium starch glycollate (Primojel).

To the excipient mixture prepared, the active ingredient granules were mixed in geometric proportion. Mixing was continued for further for 30-45 mins so as to ensure uniform distribution of the granules in the excipient mix.

Tablets were punched using single a concave or capsule shaped die punch. Thus, coloured, circular, biconvex or capsule shaped tablets with a sweet odor with a hardness of 5-7 kg/cm² and a friability value of 0.2% were obtained. The dispersible tablets disintegrated within less than 1 minute when brought in contact with water at room temperature.

### Disintegration Time:

The disintegration test was carried out according to I.P. The tablet was placed in a disintegration tester containing around 800 ml of water at room temperature. The tablet disintegrated within less than 1 min (in 45 sec.)

The following examples are provided to further illustrate the invention with specific embodiments stipulated in the present invention. The scope of the present invention is not restricted to amoxicillin but is intended to include any other class of antibiotics.

### EXAMPLE 1

| Ingredients | gm/tablet |
|---|---|
| Amoxicillin trihydrate | 0.2295 |
| Ethyl Cellulose | 0.1722 |
| Primojel | 0.151 |
| Aerosil | 0.091 |
| Erythrosine | 0.002 |
| Strawberry flavor | 0.005 |
| Sodium saccharin | 0.018 |

### EXAMPLE 2

| Ingredients | gm/tablet |
|---|---|
| Amoxicillin trihydrate | 0.2639 |
| Ethyl Cellulose | 0.2639 |
| Primojel | 0.151 |
| Aerosil | 0.091 |
| Erythrosine | 0.002 |
| Strawberry flavor | 0.005 |
| Sodium saccharin | 0.018 |

### EXAMPLE 3

| Ingredients | gm/tablet |
|---|---|
| Amoxicillin trihydrate | 0.2984 |
| Ethyl Cellulose | 0.373 |
| Primojel | 0.151 |
| Aerosil | 0.091 |
| Erythrosine | 0.002 |
| Strawberry flavor | 0.005 |
| Sodium saccharin | 0.018 |

### EXAMPLE 4

| Ingredients | gm/tablet |
|---|---|
| Amoxicillin trihydrate | 0.396 |
| Ethyl Cellulose | 0.792 |
| Primojel | 0.151 |
| Aerosil | 0.091 |
| Erythrosine | 0.002 |
| Strawberry flavor | 0.005 |
| Sodium saccharin | 0.018 |

### EXAMPLE 5

| Ingredients | gm/tablet |
|---|---|
| Amoxicillin trihydrate | 0.2869 |
| Ethyl Cellulose | 0.2152 |
| Primojel | 0.151 |
| Aerosil | 0.091 |
| Erythrosine | 0.002 |
| Strawberry flavor | 0.005 |
| Sodium saccharin | 0.018 |

### EXAMPLE 6

| Ingredients | gm/tablet |
|---|---|
| Amoxicillin trihydrate | 0.5763 |
| Ethyl Cellulose | 0.4322 |
| Primojel | 0.151 |
| Aerosil | 0.091 |
| Erythrosine | 0.002 |
| Strawberry flavor | 0.005 |
| Sodium saccharin | 0.018 |

### Examples for Cefixime Trihydrate Dispersible Sustained Release Tablets

### EXAMPLE 7

| Ingredients | gm/tablet |
|---|---|
| Cefixime trihydrate | 0.2238 |
| Ethyl Cellulose | 0.1119 |
| Primojel | 0.151 |
| Aerosil | 0.091 |
| Erythrosine | 0.002 |
| Strawberry flavor | 0.005 |
| Sodium saccharin | 0.018 |

### EXAMPLE 8

| Ingredients | gm/tablet |
|---|---|
| Cefixime trihydrate | 0.2238 |
| Ethyl Cellulose | 0.1678 |
| Primojel | 0.151 |
| Aerosil | 0.091 |
| Erythrosine | 0.002 |
| Strawberry flavor | 0.005 |
| Sodium saccharin | 0.018 |

### EXAMPLE 9

| Ingredients | gm/tablet |
|---|---|
| Cefixime trihydrate | 0.2238 |
| Ethyl Cellulose | 0.2238 |
| Primojel | 0.151 |
| Aerosil | 0.091 |
| Erythrosine | 0.002 |
| Strawberry flavor | 0.005 |
| Sodium saccharin | 0.018 |

### EXAMPLE 10

| Ingredients | gm/tablet |
|---|---|
| Cefixime trihydrate | 0.4476 |
| Ethyl Cellulose | 0.2239 |
| Primojel | 0.175 |
| Aerosil | 0.12 |
| Erythrosine | 0.002 |
| Strawberry flavor | 0.0075 |
| Sodium saccharin | 0.02 |

### EXAMPLE 11

| Ingredients | gm/tablet |
|---|---|
| Cefixime trihydrate | 0.4476 |
| Ethyl Cellulose | 0.3357 |
| Primojel | 0.175 |
| Aerosil | 0.12 |
| Erythrosine | 0.002 |
| Strawberry flavor | 0.0075 |
| Sodium saccharin | 0.02 |

### EXAMPLE 12

| Ingredients | gm/tablet |
|---|---|
| Cefixime trihydrate | 0.4476 |
| Ethyl Cellulose | 0.4476 |
| Primojel | 0.175 |
| Aerosil | 0.12 |
| Erythrosine | 0.002 |
| Strawberry flavor | 0.0075 |
| Sodium saccharin | 0.02 |

### EXAMPLE 13

| Ingredients | gm/tablet |
|---|---|
| Cefixime trihydrate | 0.4476 |
| Ethyl Cellulose | 0.2239 |
| Primojel | 0.2 |
| Aerosil | 0.12 |
| Erythrosine | 0.002 |
| Strawberry flavor | 0.0075 |
| Sodium saccharin | 0.02 |

### EXAMPLE 14

| Ingredients | gm/tablet |
|---|---|
| Cefixime trihydrate | 0.4476 |
| Ethyl Cellulose | 0.3357 |
| Primojel | 0.2 |
| Aerosil | 0.12 |
| Erythrosine | 0.002 |
| Strawberry flavor | 0.0075 |
| Sodium saccharin | 0.02 |

### EXAMPLE 15

| Ingredients | gm/tablet |
|---|---|
| Cefixime trihydrate | 0.4476 |
| Ethyl Cellulose | 0.4476 |
| Primojel | 0.2 |
| Aerosil | 0.12 |
| Erythrosine | 0.002 |
| Strawberry flavor | 0.0075 |
| Sodium saccharin | 0.02 |

### Dissolution Testing Methods:

The release of amoxicillin from the tablets was determined using the Dissolution Rate Test Apparatus 1, official in USP 22, by pH change method.

### Test Specifications:

Temperature: Room temp
Dissolution medium: pH 1.2 buffer for the first 2 hours, 900 ml.
pH 7.2 buffer for the remaining period, 900 ml

### Basket Speed: 100 rpm

### Method:

Tablet was tied in a nylon cloth and kept in the basket which was then immersed in the dissolution medium. Aliquots of medium were removed after 30 mins, 1 h, 12 h, 2 h, 3 h, 4 h, 5 h, 6 h, 7 h, 8 h, each aliquot being replaced simultaneously by an equal volume of medium to maintain constant volume. The amount of active substance was determined by UV spectrometry, at 227 nm for both the buffers.

### Dissolution Release Profile for the Examples 1 to 6:

| | % active ingredient released | | | | | |
|---|---|---|---|---|---|---|
| Time | | | Exam- | | | Exam- |
| in h | Example 1 | Example 2 | ple 3 | Example 4 | Example 5 | ple 6 |
| 0 | 0 | 0 | 0 | 0 | 0 | 0 |
| 0.5 | 18.09 | 17.03 | 17.91 | 19.52 | 13.70 | 17.37 |
| 1 | 24.19 | 24.74 | 25.29 | 26.34 | 21.36 | 24.15 |
| 2 | 37.11 | 39.58 | 39.13 | 39.50 | 33.85 | 33.97 |
| 3 | 47.84 | 52.82 | 51.35 | 50.50 | 44.85 | 40.90 |
| 4 | 51.57 | 54.36 | 55.97 | 53.89 | 48.74 | 41.94 |
| 5 | 54.44 | 58.60 | 58.64 | 56.63 | 51.52 | 43.21 |
| 6 | 56.55 | 57.91 | 62.55 | 59.84 | 50.92 | 43.97 |
| 7 | 58.69 | 59.40 | 62.92 | 62.35 | 52.66 | 45.07 |
| 8 | 59.79 | 60.77 | 65.25 | 63.93 | 55.00 | 46.42 |

### In Vivo Evaluation of Test Formulation

A comparison of bioavailability and other pharmacokinetic parameters of amoxicillin after administration of sustained release dispersible tablets and that of a commercial preparation was taken up to ascertain in vivo performance of developed formulation and compare it with a conventional product. Rabbit was used as an animal model.

### Protocol:

Three adult rabbits of either sex, weighing between 1.5 to 2.5 kg were taken for the study. All the three rabbits received both the formulations. On the first day of the trial, the rabbits were given the test formulation. After a wash-out period of 7 days, the rabbits were given the commercial formulation.

Before administering the dosage form, control blood samples of 1 ml were obtained from the marginal ear vein of all the rabbits. At zero time, the formulation was administered. Blood samples (1 ml each) were then obtained at time intervals 0.5, 1, 1.5, 2, 4, 6, 8 and 24 h after the administration of the dose.

Each of the blood sample was collected in the test tube containing 0.3 ml of 3.8% w/v sodium citrate. The blood samples were centrifuged within 30 mins after collection, and the plasma was separated and stored at −20° C. until analysis. Amoxicillin was assayed by the agar well diffusion method.

Pharmacokinetic Parameters of Amoxicillin in Rabbit After Oral Administration of Test and Commercial Formulation

| | | Cₘₐₓ | AUC_{0-24 h} | |
|---|---|---|---|---|
| Formulation | Rabbit | (μg/ml) | (μg · h/ml) | MRT (h) |
| Commercial | 1. | 9.21 | 26.17 | 4.2 |
| Sample | 2. | 8.79 | 24.83 | 4.4 |
| | 3. | 8.26 | 24.47 | 4.5 |
| | Mean ± | 8.75 ± 0.38 | 25.16 ± 0.73 | 4.4 ± 0.12 |
| | S.D. | | | |
| Test Formulation | 1. | 6.23 | 44.16 | 9.8 |
| | 2. | 5.25 | 44.01 | 10.3 |
| | 3. | 5.77 | 44.61 | 10.7 |
| | Mean ± | 5.75 ± 0.4 | 44.30 ± 0.25 | 10.25 ± 0.34 |
| | S.D. | | | |

### Statistical Analysis of the Pharmacokinetic Parameters Calculated for the Amoxicillin Test Formulation and Commercial Formulation

| | | 5% | Analysis of | 5% |
|---|---|---|---|---|
| | Student's | significance | variance | significance |
| Variable | t-test | level | (ANOVA) | level |
| Drug plasma conc (μg/ml) in | 2.21 | Significant | — | — |
| commercial and test | | | | |
| formulations | | | | |
| AUC_{0-24 h} (μg · h/ml) in | 6.07 | Significant | — | — |
| commercial and test | | | | |
| formulations | | | | |
| Drug plasma conc (μg/ml) in | — | — | 4.89 | Significant |
| commercial formulation, | | | | |
| within the 3 rabbits | | | | |
| Drug plasma conc (μg/ml) in | — | — | 2.24 | Not |
| test formulation, within the 3 | | | | Significant |
| rabbits | | | | |
| AUC_{0-24 h} (μg · h/ml) in | — | — | 5.02 | Significant |
| commercial formulation, within | | | | |
| the 3 rabbits | | | | |
| AUC_{0-24 h} (μg · h/ml) in test | — | — | 0.107 | Not |
| formulation, within the 3 | | | | Significant |
| rabbits | | | | |

The drug from the test formulation of amoxicillin was absorbed rapidly within half-an-hour giving a peak concentration of 5.75 μg/ml. Drug plasma concentration declined gradually with time. The concentration at the end of 2 h was 2.09 μg/ml and the levels were maintained between 1.87-1.8 μg/ml from the 4^{th} h till the 8^{th} h. Significant levels of amoxicillin (1.43 μg/ml) were present in plasma after 24 h. Analysis of variance (ANOVA) at 5% significance level, indicated no significant difference in the plasma concentration and the (AUC) within the rabbits.

As compared to test formulation, commercial formulation showed a mean peak concentration of 8.75 μg/ml at half-an-hour post administration after which the levels declined rapidly. The concentration at the end of 2 h was 2.62 μg/ml and at the end of 8 h a drug plasma concentration of 1.03 μg/ml was obtained. Blood sample withdrawn at 24 h showed no amoxicillin.

Student's t-test, at 5% significance level, indicated a significant difference in the plasma drug concentration and the AUC between the test and the commercial formulations.

The plasma concentration data of both the commercial and test a formulation was evaluated by statistical moment analysis in terms of mean residence time (MRT). MRT is calculated as:

$$MRT = \frac{AUMC}{AUC}$$

where AUMC is the area under the ‘first moment curve’ and is obtained from a plot of the product of drug concentration in plasma and time v/s time. AUC is the area under the ‘zero moment curve’ and is obtained by plotting the drug concentration in plasma v/s time.

It was observed that MRT of the test formulation was 10.25 h and that of the commercial formulation was 4.4 h. An increase of about 6 h was observed in the MRT of the test formulation as compared to the commercial formulation indicating a sustained effect of the test formulation.

High plasma amoxicillin levels do not correspond to increased efficacy of the drug. Though low drug levels in blood seem to indicate poor bioavailability, this however has no relevance from the therapeutic point of view. The important factor is the time period over which the drug levels are maintained above the minimum inhibitory concentration (MIC).

The test formulation maintained the drug levels above the MIC for a longer period of time than the commercial formulation. MIC (0.25 μg/ml) for the susceptible pathogen was maintained up to 24 h by the test formulation as compared to the commercial formulation. Also the commercial formulation gave too high Cₘₐₓ values which are not desirable and may lead to side effects of the drug.

It can thus be concluded that the developed tablet formulation maintained the drug levels for a longer period of time and had a mean residence time of 10.25 h and thus showed a significant sustained action as compared to the commercial formulation.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

In the claims appended, we use the singular to encompass the plural. For example, the phrase “polymer selected from the group consisting of A, B and C” covers compositions with one of the enumerated polymers, or two of them, or even all three together.

We claim:

1. A dispersible sustained release tablet composition comprising sustained release granules of at least one active pharmaceutical ingredient and at least one release retard hydrophobic polymer, a super disintegrant and a lubricant.

2. The dispersible sustained release tablet composition as claimed in claim 1 wherein said release retard hydrophobic polymer is selected from methyl cellulose, ethylcellulose, Carbomers, Eudragits, Hydroxy propyl methyl cellulose, Hydroxy propyl cellulose, Hydroxy ethyl cellulose or ethyl cellulose.

3. The dispersible sustained release tablet composition as claimed in claim 1, wherein said active pharmaceutical ingredient and the release retard polymer ratio is in the range of 1:0.5 to 1:3, preferably in the ratio of 1:0.75.

4. The dispersible sustained release tablet composition as claimed in claim 1, wherein said granules are prepared by wet granulation technique.

5. The dispersible sustained release tablet composition as claimed in claim 1, wherein said active pharmaceutical ingredient is antibiotic.

6. The dispersible sustained release tablet composition as claimed in any of the preceding claims, wherein said active pharmaceutical ingredient is selected from the group of β-lactams, cephalosporins or penicillins.

7. The dispersible sustained release tablet composition as claimed in claim 1, wherein said super disintegrant is selected from croscarmellose sodium, crospovidone, primojel, sodium starch glycolate or starch, in an amount of 15 to 25% of the tablet weight.

8. The dispersible sustained release tablet composition as claimed in claim 1, wherein said lubricant is selected from the group of metallic silicates, metallic stearates starch, talc, micronized amorphous silica, amorphous silica or colloidal silicon dioxide (Aerosil), used in amount of 5-15%.

9. The dispersible sustained release tablet composition as claimed in claim 1 further comprises coloring agent in an amount of 0.3 to 0.8% of the tablet weight.

10. The dispersible sustained release tablet composition as claimed in claim 1, further comprises flavoring agent in an amount of 0.3 to 0.8% of the tablet weight.

11. The dispersible sustained release tablet composition as claimed in claim 1, further comprises suitable sweetening agents in an amount of 1.0 to 3.0% of tablet weight.

12. The dispersible sustained release tablet composition as claimed in claim 1 wherein said granules are able to sustain the release of the active ingredient over a period of 12 hrs in gastro intestinal tract.

13. The dispersible sustained release tablet composition as claimed in claim 1 wherein said active pharmaceutical ingredient is amoxicillin trihydrate.

14. The dispersible sustained release tablet composition as claimed in claim 1 wherein said active pharmaceutical ingredient is cefixime trihydrate.
